(19) United States
(12) Patent Application Publication
(10) Pub. No.: US 2005/0126200 A1
(43) Pub. Date: Jun. 16, 2005

(54) Single valve manifold

(76) Inventors: Ajit Ramachandran, Fort Wayne, IN (US); Keith Sliger, Corunna, IN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 11/003,946

(22) Filed: Dec. 3, 2004

Related U.S. Application Data
(60) Provisional application No. 60/527,241, filed on Dec. 5, 2003.

(51) Int. Cl. A61M 37/00; F25B 45/00

(52) U.S. Cl. 62/292

(57) ABSTRACT

A valve manifold assembly may comprise an outer casing, a first valve portion and a second valve portion. A first valve portion may comprise a plurality of fluid inlet orifices, a fluid outlet orifice and a first valve member having a first fluid pathway. A second valve portion may comprise a plurality of fluid inlet orifices, a plurality of fluid outlet orifices and a second valve member having a second fluid pathway. The valve members may be fixedly attached to a rotatable spindle having a plurality of stop positions, each stop position placing the valve fluid pathways in fluid communication with various inlet and outlet orifices.

### CROSS-REFERENCE TO RELATED APPLICATIONS

This application claims priority to U.S. Provisional Patent Application No. 60/527,241 filed Dec. 5, 2003, the entire contents of which is incorporated herein by reference in its entirety.

### BACKGROUND OF THE INVENTION

This invention relates generally to a valve assembly and more specifically to a valve assembly that may be used in a refrigerant recovery and reclaim machine.

Refrigerant recovery and reclaim machines and their use are known in the art. These machines generally include a plurality of valves and hose connections which must be operated in a predetermined sequence in order to achieve proper operation of the machine. The operation of valves and the rearrangement of hose connections may be subject to error when the predetermined procedure is not followed exactly. Errors may cause problems ranging from inefficient use of time to the undesirable release of refrigerant into the atmosphere.

There remains a need for a device which simplifies the process of recovering refrigerant and is less prone to operator error than existing recovery and reclaim machines.

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In one embodiment, a manifold for a refrigerant handling apparatus may comprise an outer casing having a plurality of fluid inlet orifices and a plurality of fluid outlet orifices. At least one conduit external to the outer casing may be arranged to provide a fluid passage between at least one of said fluid inlet orifices and at least one of said fluid outlet orifices. At least one valve may be rotatably disposed within the outer casing and may include at least one traverse port for fluid flow communication with at least one of said fluid outlet orifices following rotation of the valve relative to the outer casing.

In another embodiment, a manifold for a refrigerant handling apparatus may comprise an outer casing having a plurality of fluid inlet orifices, a plurality of fluid outlet orifices, and at least one external conduit in fluid flow communication between at least one of said fluid inlet orifices and at least one of said fluid outlet orifices. A plurality of valves may be rotatably disposed within the outer casing. Each of the valves may comprise a plurality of traverse ports. Each of the traverse ports for each valve may be constructed and arranged to be in fluid flow communication to another of said traverse ports. At least one of said traverse ports for each of the valves may be constructed and arranged for fluid flow communication with at least one of the fluid inlet orifices. At least one of the traverse ports for each of the valves may be constructed and arranged for fluid flow communication with at least one of the fluid outlet orifices. A spindle may be connected to each of the valves and may extend from one of the valves through the outer casing. An actuator may be engaged to the spindle and arranged for rotation of the spindle and the plurality of valves relative to the outer casing. At least one of the traverse ports may be positioned proximate to at least one of the fluid inlet orifices.

In another embodiment, a valve assembly may comprise a casing having a first portion and a second portion, a first valve member and a second valve member. The first portion of the casing may define a first internal cavity and may have a first aperture, a second aperture and a third aperture. The second portion of the casing may define a second internal cavity and may have a fourth aperture, a fifth aperture, a sixth aperture and a seventh aperture. The first valve member may be oriented within the first internal cavity and may define a first valve fluid passageway, and the second valve member may be oriented within the second internal cavity and may define a second valve fluid passageway. A rotatable spindle may be coupled to the first valve member and the second valve member and may be arranged to rotate the first valve member and the second valve member. The rotatable spindle may include at least two stop positions. When the rotatable spindle is in a first stop position, the first valve fluid passageway may be oriented to allow fluid flow between the first aperture and the second aperture, and the second valve fluid passageway may be oriented to allow fluid flow between the fourth aperture and the fifth aperture.

These and other embodiments which characterize the invention are pointed out with particularity in the claims annexed hereto and forming a part hereof. However, for a better understanding of the invention, its advantages and objectives obtained by its use, reference should be made to the drawings which form a further part hereof and the accompanying descriptive matter, in which there are illustrated and described various embodiments of the invention.

### BRIEF DESCRIPTION OF THE DRAWINGS

A detailed description of the invention is hereafter described with specific reference being made to the drawings.

FIG. 1 shows an embodiment of a valve manifold assembly.

FIG. 2 shows an exploded view of an embodiment of a valve manifold assembly.

FIG. 3 shows a longitudinal cross-sectional view of an embodiment of a valve manifold assembly.

FIG. 4 shows a recovery and reclaim device schematic using an embodiment of a valve manifold assembly.

FIG. 5 shows a recovery and reclaim device schematic using an embodiment of a valve manifold assembly during a recovery operation.

FIG. 6 shows a recovery and reclaim device schematic using an embodiment of a valve manifold assembly during a stop/prepurge operation.

FIG. 7 shows a recovery and reclaim device schematic using an embodiment of a valve manifold assembly during a purge operation.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

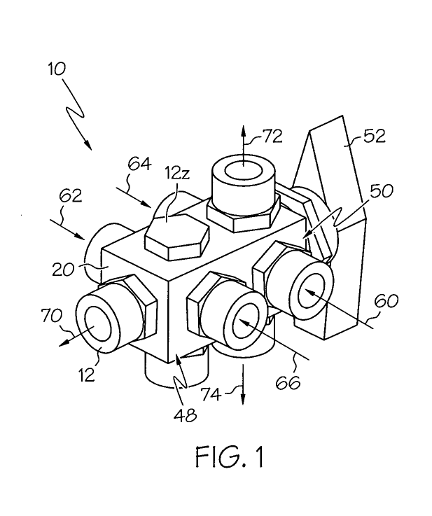

Referring to FIGS. 1-3, an inventive valve manifold assembly 10 is depicted, which may comprise an outer casing 20, a plurality of fittings 12, a first valve member 30, a second valve member 40 and a rod or spindle 18.

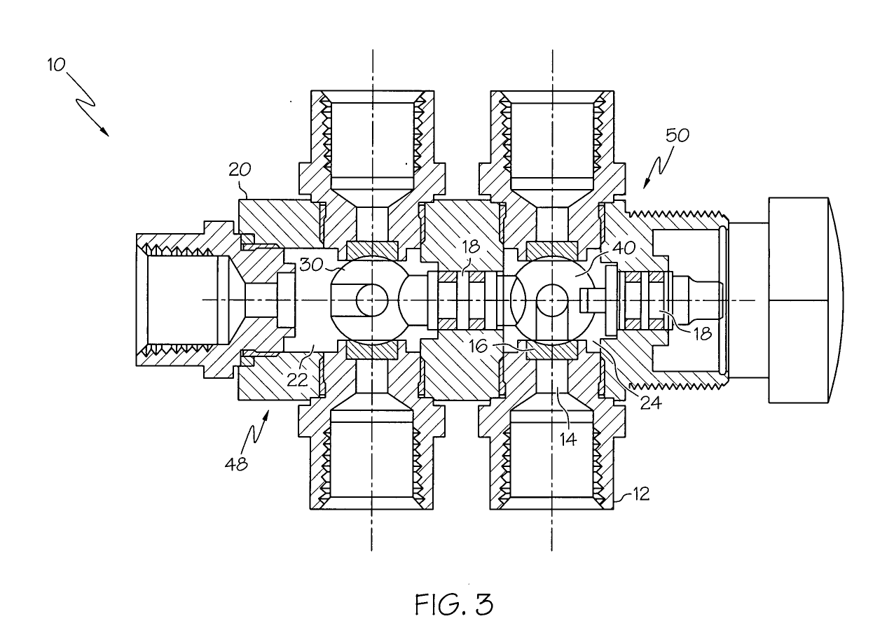

The outer casing 20 may be made from any suitable material, such as brass, bronze, steel, aluminum, plastics, resin and/or any other desired material, and preferably defines a first internal cavity 22 (see FIG. 3) and a second internal cavity 24. A plurality of apertures 28 may extend through the various wall portions of the outer casing 20. Each aperture 28 may be constructed and arranged to receive and engage a fitting 12. For example, a fitting 12 may be machined to include threadings which may engage a threaded aperture 28.

The first valve member 30 and the second valve member 40 may each be fixed to the spindle 18 and thus may be arranged to rotate with the spindle 18. The first valve member 30 may be oriented within the first internal cavity 22 of the outer casing 20, and the second valve member 40 may be oriented within the second internal cavity 24 of the outer casing 20.

Each fitting 12 may include a fluid passageway 14 extending from one end of the fitting 12 to the other. Each fitting 12 may be arranged to abut a valve member 30, 40 and may thus include a seat or sealing member 16 at one end. A sealing member 16 may comprise any suitable shape and be made of any suitable material capable of sealing against a valve member 30, 40. For example, a sealing member 16 may comprise a nylon ring. In embodiments where a valve member 30, 40 comprises a ball or includes a generally spherical shape, a portion of the sealing member 16 may include a spheroidal shape arranged to properly engage the outer surface of the ball. Each fitting 12, at the side opposite the sealing member 16, may be arranged to engage a bushing, T-fitting, hose or other fluid conduit, for example by the use of threadings or other coupling members.

It should be understood that the valve members 30, 40 are not limited to substantially spherical shapes. Valve members 30, 40 may have any suitable shape which provides for proper operation of the valve manifold assembly 10. For example, valve members 30, 40 may alternatively have a cylindrical shape and be rotatable about a longitudinal axis. Further, the shape of the first valve member 30 may differ from the shape of the second valve member 40 in some embodiments.

It should also be understood that fittings 12 are not required to be used. For example, in some embodiments, various apertures 28 in the outer casing 20 may be provided with a valve seat, and a valve member 30, 40 may abut the apertures 28 and may be contained between wall portions of the outer casing 20.

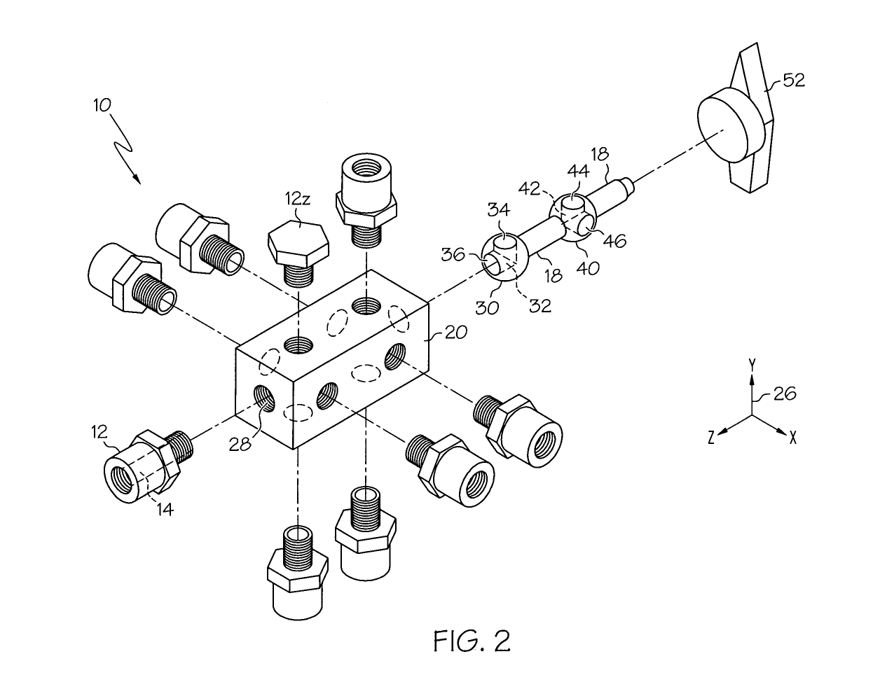

In some embodiments, some fittings 12 are not required to include a fluid passageway 14, and therefore may comprise a plug. For example, fitting 12z as depicted in FIGS. 1 and 2 comprises a plug. A plug may be arranged to engage the outer casing 20 and may include a seat or sealing member 16 which abuts a valve member 30, 40. A plug may be used in locations where the specific embodiment of the valve manifold assembly 10 is not required to include a fluid passageway at the location of the fitting 12.

An actuator 52 may be engaged to the spindle 18 or may otherwise be engaged to the valve members 30, 40. The actuator 52 may control actuation or rotation of the valve members 30, 40 through a plurality of stop positions. The actuator 52 and/or the valve members 30, 40 may be predisposed to stopping at any or all of the stop positions, for example by the use of a notched detent. In some embodiments, an actuator 52 may comprise a handle which may be operated by a technician. In some embodiments, an actuator 52 may comprise a motor, servomotor or the like, which may be operated by a computer or via an interface. In some embodiments, an interface may include a momentary switch or button for each stop position, and operation of a given momentary switch or button may cause the actuator 52 and valve members 30, 40 to assume an appropriate stop position orientation.

The first valve member 30 may include a first fluid pathway 32 which may comprise a first inlet traverseport 34 and a first outlet traverseport 36. The second valve member 40 may include a second fluid pathway 42 which may comprise a second traverseport 44 and a third traverseport 46.

Rotation of the valve members 30, 40, for example via the actuator 52, will align the various traverseports 34, 36, 44, 46 of the valve members 30, 40 with the fluid passageways 14 of various fittings 12 at each desirable stop position as described below.

Referring again to FIG. 1, with respect to one embodiment of a valve manifold assembly 10, the pathways provided for fluid flow will be identified individually. In some embodiments, the pathways may be provided through fittings 12. In some embodiments, for example when fittings 12 are not used, the pathways may be provided through the outer casing 20 or any other suitable portion of the device. The valve manifold assembly 10 may be provided with a first fluid inlet orifice 60, a second fluid inlet orifice 62, a third fluid inlet orifice 64, a fourth fluid inlet orifice 66, a first fluid outlet orifice 70, a second fluid outlet orifice 72 and a third fluid outlet orifice 74.

The valve manifold assembly 10 may define a first valve portion 48 and a second valve portion 50. The first valve portion 48 may comprise the first valve member 30, and may further include the second fluid inlet orifice 62, the fourth fluid inlet orifice 66 and the first fluid outlet orifice 70. The fluid orifices 62, 66, 70 provided for the first valve portion 48 may or may not be in fluid communication with the first fluid pathway 32 of the first valve member 30 at various rotational orientations of the first valve member 30, as will be described with respect to operation of the valve manifold assembly 10. The second valve portion 50 may comprise the second valve member 40, and may further include the first fluid inlet orifice 60, the third fluid inlet orifice 64, the second fluid outlet orifice 72 and the third fluid outlet orifice 74. The fluid orifices 60, 64, 72, 74 provided for the second valve portion 50 may or may not be in fluid communication with the second fluid pathway 42 of the second valve member 40 at various rotational orientations of the second valve member 40, as will be described with respect to operation of the valve manifold assembly 10.

Referring to FIG. 2, the shape and orientation of the first fluid pathway 32 within the first valve member 30 and the second fluid pathway 42 within the second valve member 40 are depicted with respect to one embodiment of the invention. The first fluid pathway 32 may comprise the first inlet traverseport 34 and the first outlet traverseport 36. The first inlet traverseport 34 may be oriented with a longitudinal axis spanning in the direction of a y-axis as shown on the coordinate axis 26. The first outlet traverseport 36 may be oriented with a longitudinal axis spanning in the direction of a z-axis. Thus, the first outlet traverseport 36 may be oriented with a longitudinal axis orthogonal to the longitudinal axis of the first inlet traverseport 34. The second fluid pathway 42 may comprise the second traverseport 44 and the third traverseport 46. The second traverseport 44 may be oriented with a longitudinal axis spanning in the direction of the y-axis and may be parallel to the first inlet traverseport 34. The third traverseport 46 may be oriented with a longitudinal axis spanning in the direction of an x-axis of the coordinate axis 26. Thus, the third traverseport 46 may be oriented with its longitudinal axis orthogonal to the longitudinal axis of the second traverseport 44 and orthogonal to the longitudinal axis of the first outlet traverseport 36.

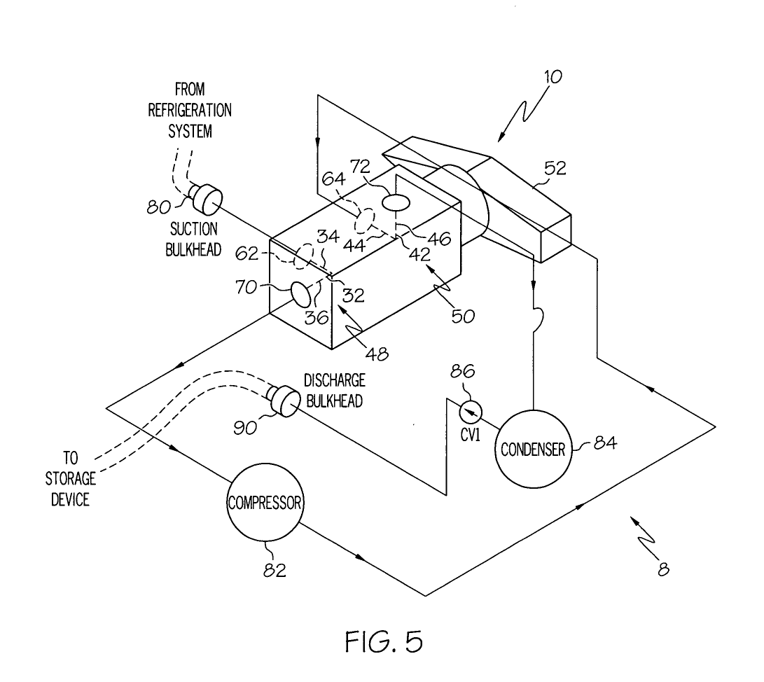

FIGS. 5-7 show the orientation of the fluid pathways 32, 42 of the valve members 30, 40 at various stop positions of the actuator 52. In a first stop position as depicted in FIG. 5, the first inlet traverseport 34 may be aligned with the second fluid inlet orifice 62 and the first outlet traverseport 36 may be aligned with the first fluid outlet orifice 70. Therefore, the first valve member 30 is oriented to allow fluid communication between the second fluid inlet orifice 62 and the first fluid outlet orifice 70. The second valve member 40 may be oriented to allow fluid communication between the third fluid inlet orifice 64 and the second fluid outlet orifice 72, as the second traverseport 44 may be aligned with the third fluid inlet orifice 64 and the third traverseport 46 may be aligned with the second fluid outlet orifice 72.

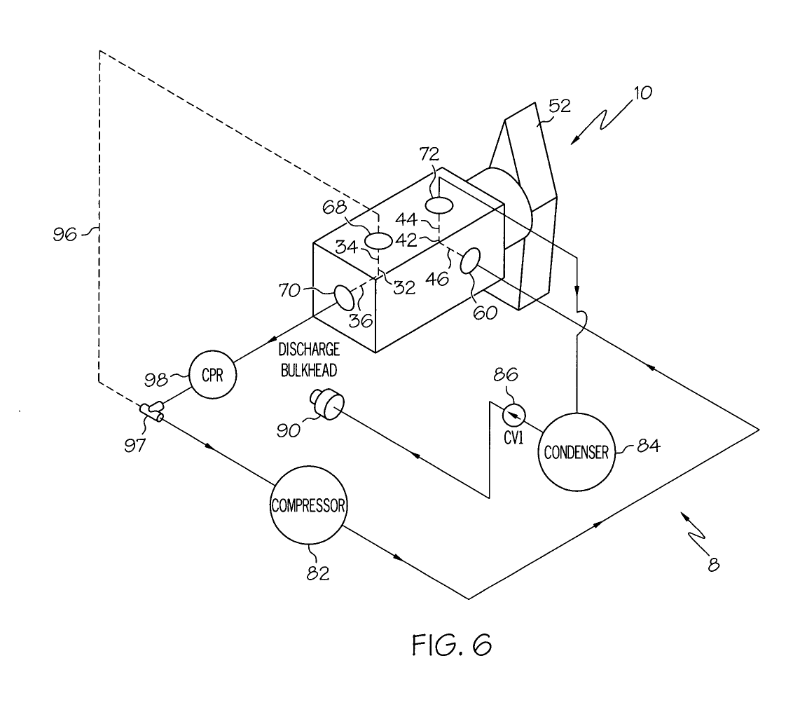

FIG. 6 depicts the orientation of the fluid pathways 32, 42 of the valve members 30, 40 at a second stop position. The orientation of the valve members 30, 40 at the second stop position may be rotated 90° from that of the first stop position. The first inlet traverseport 34 may be aligned with an aperture 28 or orifice 68 that is not used in some embodiments. The first outlet traverseport 36 may be aligned with the first fluid outlet orifice 70. The second valve member 40 may be oriented to allow fluid communication between the first fluid inlet orifice 60 and the second fluid outlet orifice 72, as the second traverseport 44 may be aligned with the second fluid outlet orifice 72 and the third traverseport 46 may be aligned with the first fluid inlet orifice 60.

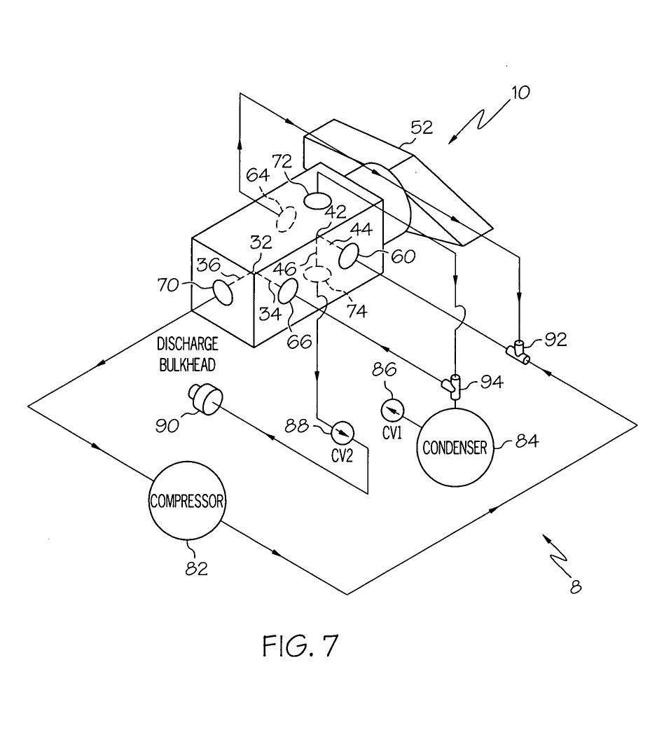

FIG. 7 depicts the orientation of the fluid pathways 32, 42 of the valve members 30, 40 at a third stop position. The orientation of the valve members 30, 40 at the third stop position may be rotated 90° from that of the second stop position and 180° from that of the first stop position. The first valve member 30 may be oriented to allow fluid communication between the fourth fluid inlet orifice 66 and the first fluid outlet orifice 70, as the first inlet traverseport 34 may be aligned with the fourth fluid inlet orifice 66 and the first outlet traverseport 36 may be aligned with the first fluid outlet orifice 70. The second valve member 40 may be oriented to allow fluid communication between the first fluid inlet orifice 60 and the third fluid outlet orifice 74, as the second traverseport 44 may be aligned with the first fluid inlet orifice 60 and the third traverseport 46 may be aligned with the third fluid outlet orifice 74.

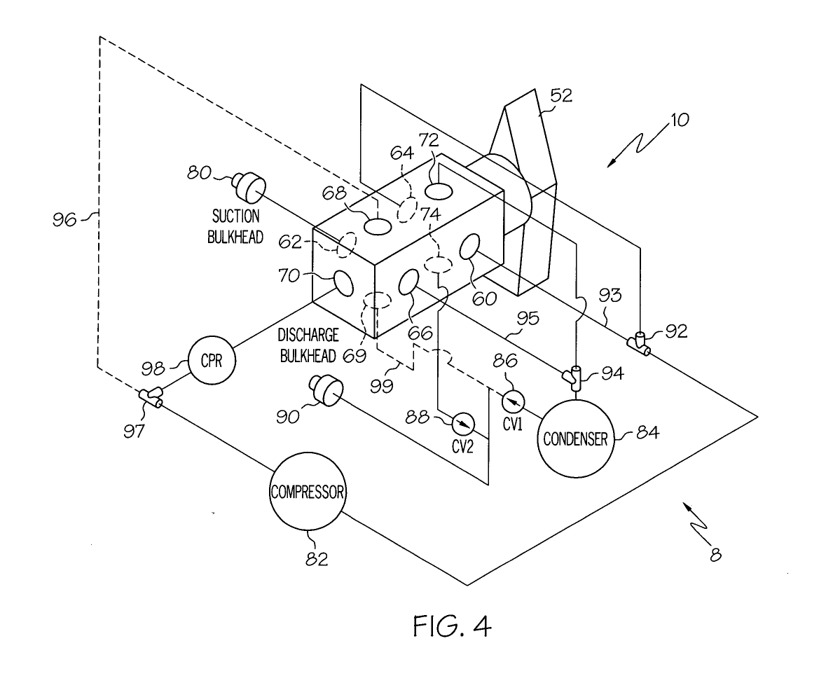

The valve manifold assembly 10 may be used as part of a recovery and reclaim device. FIG. 4 depicts a recovery and reclaim device 8 which uses an embodiment of the valve manifold assembly 10. The second fluid inlet orifice 62 may be in fluid communication with a suction bulkhead 80. The first fluid outlet orifice 70 may be in fluid communication with the intake side of a compressor 82. The output side of the compressor 82 may be in fluid communication with the third fluid inlet orifice 64. The first fluid inlet orifice 60 may also be in fluid communication with the output side of the compressor 82, for example by using a first external fluid conduit 93 and a first T-fitting 92. The second fluid outlet orifice 72 may be in fluid communication with an input side of a condenser 84. The fourth fluid inlet orifice 66 may also be in fluid communication with the second fluid outlet orifice 72, for example by using a second external fluid conduit 95 and a second T-fitting 94. An output side of the condenser 84 may be in fluid communication with a discharge bulkhead 90. Preferably, a first check valve 86 may be used between the condenser 84 and the discharge bulkhead 90 which may be oriented to allow flow only from the condenser 84 to the discharge bulkhead 90. The third fluid outlet orifice 74 may also be placed in fluid communication with the discharge bulkhead 90, preferably using a second check valve 88 oriented to allow flow only from the third fluid outlet orifice 74 to the discharge bulkhead 90.

In some embodiments, a first pressure equalization orifice 68 may be used. A first pressure equalization orifice 68 may be positioned such that the first pressure equalization orifice 68 is in fluid communication with the first fluid outlet orifice 70 when the first valve member 30 is at the second stop position. The first pressure equalization orifice 68 may be used in conjunction with a Constant Pressure Regulator (CPR) valve 98 installed between the first fluid outlet orifice 70 and the intake side of the compressor 82. The CPR valve 98 may be arranged to receive refrigerant from the first fluid outlet orifice 70, flash liquid refrigerant to vapor and regulate the pressure of vapor reaching the compressor 82. A third external fluid conduit 96 may connect at one end to the first pressure equalization orifice 68 and at the other end to the fluid line running between the CPR valve 98 and the compressor 82, for example using a third T-fitting 97. When the first valve member 30 is at the second stop position, pressure on opposite sides of the CPR valve 98 may be equalized.

A recovery and reclaim device 8 having an embodiment of the valve manifold assembly 10 may be used to recover refrigerant from a mechanical refrigeration system such as a refrigerator, air conditioner, etc. FIGS. 5-7 depict an embodiment of a recovery and reclaim device 8 during various stages of a recovery operation. A port of the refrigeration system (not shown) may be connected to the suction bulkhead 80 of the recovery and reclaim device 8, and a storage device (not shown) may be connected to the discharge bulkhead 90.

FIG. 5 shows the actuator 52 and the fluid pathways 32, 42 of the valve members at a first stop position, where the first fluid pathway 32 may connect the second fluid inlet orifice 62 and the first fluid outlet orifice 70, and the second fluid pathway 42 may connect the third fluid inlet orifice 64 and the second fluid outlet orifice 72. The first stop position may be used in a recovery operation, wherein the first valve portion 48 of the valve manifold assembly 10 may place the suction bulkhead 80 in fluid communication with the suction side of the compressor 82, and the second valve portion 50 of the valve manifold assembly 10 may place the output side of the compressor 82 in fluid communication with the input side of the condenser 84. Refrigerant may be drawn from the refrigeration system through the suction bulkhead 80, second fluid inlet orifice 62, first fluid pathway 32, first fluid outlet orifice 70, compressor 82, third fluid inlet orifice 64, second fluid pathway 42, second fluid outlet orifice 72, condenser 84, first check valve 86 and dispensed through the discharge bulkhead 90.

After a desired amount of refrigerant has been removed from the refrigeration system during a recovery operation, the recovery and reclaim device 8 may be configured for a stop/prepurge operation as depicted in FIG. 6. The actuator 52 and valve members may be oriented in the second stop position, wherein the first fluid pathway 32 may connect the first pressure equalization orifice 68 and the first fluid outlet orifice 70, and the second fluid pathway 42 may connect the first fluid inlet orifice 60 and the second fluid outlet orifice 72. Refrigerant in the recovery and reclaim device 8 may be drawn from the first fluid pathway 32 through the first fluid outlet orifice 70, compressor 82, first fluid inlet orifice 60, second fluid pathway 42, second fluid outlet orifice 72, condenser 84, first check valve 86 and dispensed through the discharge bulkhead 90.

In some embodiments, a CPR valve 98 may be installed between the first fluid outlet orifice 70 and the intake side of the compressor 82. Preferably, a third external fluid conduit 96 may be used to allow fluid communication between the first pressure equalization orifice 68 and a line between the CPR valve 98 and the compressor 82. During a stop/prepurge operation, pressure on opposite sides of the CPR valve 98 may be equalized. High pressure remaining between the first fluid outlet orifice 70 and the CPR valve 98 is allowed to pass back through the first fluid outlet orifice 70, first fluid pathway 32, first pressure equalization orifice 68, third external fluid conduit 96 and reach the downstream side of the CPR valve 98 when the first valve member 30 is placed in the second stop position.

FIG. 7 shows an embodiment of the valve manifold assembly 10 oriented for a purge operation. The actuator 52 and valve members may be oriented in the third stop position, wherein the first fluid pathway 32 may connect the fourth fluid inlet orifice 66 and the first fluid outlet orifice 70, and the second fluid pathway 42 may connect the first fluid inlet orifice 60 and the third fluid outlet orifice 74. The fluid path provided allows for fluid communication between the first check valve 86, the condenser 84, the fourth fluid inlet orifice 66 and the second fluid outlet orifice 72 via the second T-fitting, the first fluid pathway 32, the first fluid outlet orifice 70, the compressor 82, the first fluid inlet orifice 60 and the third fluid inlet orifice 64 via the first T-fitting 92, the second fluid pathway 42, the third fluid outlet orifice 74, the second check valve 74 and the discharge bulkhead 90.

Referring again to FIG. 4, in some embodiments, a second pressure equalization orifice 69 may be used to equalize pressure in the recovery and reclaim device 8. For example, in embodiments where a CPR valve 98 and the first pressure equalization orifice 68 are not used, it would be desirable to place the second pressure equalization orifice 69 in fluid communication with the downstream side of the first check valve 86, for example by using a fourth external fluid conduit 99. When the actuator 52 and valve members 30, 40 are placed into a fourth stop position, the first fluid pathway 32 may allow fluid communication between the second pressure equalization orifice 69 and the first fluid outlet orifice 70. Pressure on the downstream side of the first check valve 86 may pass through the second pressure equalization orifice 69, the first fluid pathway 32 and the first fluid outlet orifice 70 to reach the intake side of the compressor 82 and equalize pressures on opposite sides of the compressor 82.

In some embodiments, an actuator 52 and the valve members 30, 40 may include a plurality of stop positions. In some embodiments, a first stop position may be oriented 90° of rotation away from a second stop position. A third stop position may be oriented 90° away from the second stop position and 180° away from the first stop position.

In some embodiments, the actuator 52 and valve members 30, 40 may be continuously rotatable about a full 360° of rotation or more. In some embodiments, the actuator 52 and valve members 30, 40 may be rotatable less than 360°. For example, all of the desired stop positions may be included in less than 360° of rotation. From a starting orientation, the actuator 52 and valve members 30, 40 may be placed at the first stop position for a recovery operation. The actuator 52 and valve members 30, 40 may be rotated in a first direction to the second stop position for a prepurge operation. The actuator 52 and valve members 30, 40 may then be rotated in the first direction to the third stop position for a purge operation. The actuator 52 and valve members 30, 40 may then be rotated in a second direction back to the starting orientation.

In some embodiments, the invention is directed to the following numbered paragraphs.
1) A valve manifold assembly comprising:
   a first valve portion comprising a first valve member having a first fluid pathway, a first valve first fluid inlet, a first valve second fluid inlet and a first valve first fluid outlet;
   a second valve portion comprising a second valve member having a second fluid pathway, a second valve first fluid inlet, a second valve second fluid inlet and a second valve first fluid outlet; and
   a rotatable spindle coupled to said first valve member and said second valve member, wherein said first valve member and said second valve member rotate with said rotatable spindle;
   wherein when the rotatable spindle is at a first rotational orientation, said first fluid pathway allows fluid communication between said first valve first fluid inlet and said first valve first fluid outlet, and said second fluid pathway allows fluid communication between said second valve first fluid inlet and said second valve first fluid outlet; and
   wherein when the rotatable spindle is at a second rotational orientation, said second fluid pathway allows fluid communication between said second valve second fluid inlet and said second valve first fluid outlet.
2) The valve manifold assembly of paragraph 1, wherein said first rotational orientation and said second rotational orientation are 90° apart.
3) The valve manifold assembly of paragraph 1, wherein when the rotatable spindle is at a second rotational orientation, said first fluid pathway allows fluid communication between a first valve equalization outlet and said first valve first fluid outlet.
4) The valve manifold assembly of paragraph 1, wherein said second valve portion further comprises a second valve second fluid outlet;
   wherein when the rotatable spindle is at a third rotational orientation, said first fluid pathway allows fluid communication between said first valve second fluid inlet and said first valve first fluid outlet, and said second fluid pathway allows fluid communication between said second valve second fluid inlet and said second valve second fluid outlet.
5) The valve manifold assembly of paragraph 4, wherein said second rotational orientation and said third rotational orientation are 90° apart.
6) The valve manifold assembly of paragraph 4, wherein said first rotational orientation and said third rotational orientation are 180° apart.
7) A recovery and reclaim device comprising:
   an intake port;
   a discharge port;
   a compressor;
   a condenser; and
   a valve manifold assembly comprising a first valve portion, a second valve portion and a rotatable spindle, the first valve portion having a first rotatable valve member, the second valve portion having a second valve member, said first valve member and said second valve member coupled to and arranged to rotate with said rotatable spindle;
   wherein when said rotatable spindle is at a first rotational orientation, said first valve portion allows fluid communication between said intake port and said compressor, and said second valve portion allows fluid communication between said compressor and said condenser.
8) The recovery and reclaim device of paragraph 7, wherein when said rotatable spindle is at a second rotational orientation, said intake port is not in fluid communication with said compressor.
9) The recovery and reclaim device of paragraph 8, wherein when said rotatable spindle is at a second rotational orientation, said second valve portion allows fluid communication between said compressor and said condenser.
10) The recovery and reclaim device of paragraph 9, wherein said second valve portion further comprises a first fluid inlet, a second fluid inlet and a fluid outlet, wherein when said rotatable spindle is at a first rotational orientation, said second valve member allows fluid communication between said first fluid inlet and said first fluid outlet.
11) The recovery and reclaim device of paragraph 10, wherein when said rotatable spindle is at a second rotational orientation, said second valve member allows fluid communication between said second fluid inlet and said first fluid outlet.
12) The recovery and reclaim device of paragraph 8, wherein said first rotational orientation and said second rotational orientation are 90° apart.
13) The recovery and reclaim device of paragraph 8, wherein when said rotatable spindle is at a third rotational orientation, said first valve portion allows fluid communication between said condenser and said compressor, and said second valve portion allows fluid communication between said compressor and said discharge port.
14) The recovery and reclaim device of paragraph 13, wherein said second rotational orientation and said third rotational orientation are 90° apart.
15) The valve manifold assembly of paragraph 14, wherein said first rotational orientation and said third rotational orientation are 180° apart.
16) A method of recovering refrigerant comprising:
   providing a recovery and reclaim device comprising an intake port; a discharge port; a compressor; a condenser; and a valve manifold assembly comprising a first valve portion, a second valve portion and a rotatable spindle, the first valve portion having a first rotatable valve member, the second valve portion having a second valve member, said first valve member and said second valve member coupled to and arranged to rotate with said rotatable spindle;
   connecting a fluid port of an external device having refrigerant to said intake port;
   connecting said discharge port to a storage device;
   placing said rotatable spindle at a first stop position wherein said first valve portion allows fluid communication between said intake port and said compressor, and said second valve portion allows fluid communication between said compressor and said condenser; and
   operating said recovery and reclaim device to move refrigerant from said external device to said storage device.
17) The method of paragraph 16, further comprising the step of placing said rotatable spindle at a second stop position wherein said intake port is not in fluid communication with said compressor.
18) The method of paragraph 17, further comprising the step of placing said rotatable spindle at a third stop position wherein said first valve portion allows fluid communication between said condenser and said compressor, and said second valve portion allows fluid communication between said compressor and said discharge port.

The above disclosure is intended to be illustrative and not exhaustive. This description will suggest many variations and alternatives to one of ordinary skill in this field of art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A manifold for a refrigerant handling apparatus comprising:
a) an outer casing, said outer casing comprising a plurality of fluid inlet orifices and a plurality of fluid outlet orifices;
b) at least one conduit external to said outer casing, said at least one conduit being constructed and arranged to provide a fluid passage between at least one of said fluid inlet orifices and at least one of said fluid outlet orifices;
c) at least one valve rotatably disposed within said outer casing, said at least one valve comprising a spindle, said at least one valve further comprising at least one traverse port, said at least one traverse port being constructed and arranged for fluid flow communication with at least one of said fluid outlet orifices following rotation of said spindle relative to said outer casing; and
d) an actuator engaged to said spindle, said actuator being constructed and arranged to rotate said spindle and said at least one valve relative to said outer casing.

2. A manifold for a refrigerant handling apparatus comprising:
a) an outer casing, said outer casing comprising a plurality of fluid inlet orifices, a plurality of fluid outlet orifices, and at least one external conduit in fluid flow communication between at least one of said fluid inlet orifices and at least one of said fluid outlet orifices;
b) a plurality of valves rotatably disposed within said outer casing, each of said valves comprising a plurality of traverse ports, each of said traverse ports for each valve being constructed and arranged to be in fluid flow communication to another of said traverse ports, at least one of said traverse ports for each of said valves being constructed and arranged for fluid flow communication with at least one of said fluid inlet orifices, at least one of said traverse ports for each of said valves being constructed and arranged for fluid flow communication with at least one of said fluid outlet orifices;
c) a spindle connected to each of said valves, said spindle extending from one of said valves through said outer casing; and
d) an actuator engaged to said spindle, said actuator being constructed and arranged for rotation of said spindle and said plurality of valves relative to said outer casing, wherein at least one of said traverse ports is positioned proximate to at least one of said fluid inlet orifices.

3. The manifold according to claim 2, said outer casing further comprising at least two external conduits wherein each of said external conduits is in fluid flow communication between at least one of said fluid inlet orifices and at least one of said fluid outlet orifices.

4. The manifold according to claim 3, wherein said spindle and said valves are rotated to a first position wherein two of said fluid outlet orifices and one of said fluid inlet orifices are constructed and arranged for fluid flow passage for repurge of said refrigerant handling apparatus.

5. The manifold according to claim 4, wherein said spindle and said valves are rotated to a second position wherein two of said fluid inlet orifices and two of said fluid outlet orifices are constructed and arranged for fluid flow passage for recovery of refrigerant for said refrigerant handling apparatus.

6. The manifold according to claim 5, wherein said spindle and said valves are rotated into a third position wherein two of said fluid inlet orifices and three of said fluid outlet orifices are constructed and arranged for fluid flow passage for purge of said refrigerant handling apparatus.

7. The manifold according to claim 6, further comprising a first valve and a second valve, said first valve comprising a first outlet traverse port and a first inlet traverse port, said second valve comprising a second traverse port and a third traverse port.

8. The manifold according to claim 7, said outer casing further comprising a first fluid outlet orifice, a first fluid inlet orifice, a second fluid inlet orifice, a second fluid outlet orifice, a third fluid inlet orifice, a third fluid outlet orifice, and a fourth fluid inlet orifice.

9. The manifold according to claim 8, further comprising a first external conduit, and a second external conduit, said first external conduit being constructed and arranged for fluid flow communication between said third fluid inlet orifice and said first fluid inlet orifice and said second external conduit being constructed and arranged for fluid flow communication between said second fluid outlet orifice and said fourth fluid inlet orifice.

10. The manifold according to claim 9, wherein said first outlet traverse port is in fluid flow communication with said first fluid outlet orifice, said first inlet traverse port is blocked, said second traverse port is in fluid flow communication with said second fluid outlet orifice, and said third traverse port is in fluid flow communication with said first fluid inlet orifice when said spindle and said valves are rotated to said first position for repurge of said refrigerant handling apparatus.

11. The manifold according to claim 10, wherein said first outlet traverse port is in fluid flow communication with said first fluid outlet orifice, said first inlet traverse port is in fluid flow communication with said second fluid inlet orifice, said second traverse port is in fluid flow communication with said third fluid inlet orifice, and said third traverse port is in fluid flow communication with said second fluid outlet orifice when said spindle and said valves are rotated to said second position for recovery of refrigerant for said refrigerant handling apparatus.

12. The manifold according to claim 11, wherein said fourth fluid inlet orifice and said third fluid inlet orifice are blocked when said spindle and said valves are rotated to said first position.

13. The manifold according to claim 12, wherein said fourth fluid inlet orifice and said first fluid inlet orifice are blocked when said spindle and said valves are rotated to said second position.

14. The manifold according to claim 13, wherein said first outlet traverse port is in fluid flow communication with said first fluid outlet orifice, said first inlet traverse port is in fluid flow communication with said fourth fluid inlet orifice, said second traverse port is in fluid flow communication with said first fluid inlet orifice, said third traverse port is in fluid flow communication with said third fluid outlet orifice, and said second external conduit is in fluid flow communication between said second fluid outlet orifice and said fourth fluid inlet orifice when said spindle and said valves are rotated to said third position for purge of said refrigerant handling apparatus.

15. The manifold according to claim 14, wherein said third fluid inlet orifice is blocked when said spindle and said valves are rotated to said third position.

16. A valve assembly comprising:
a casing having a first portion and a second portion; the first portion defining a first internal cavity and having a first aperture, a second aperture and a third aperture; the second portion defining a second internal cavity and having a fourth aperture, a fifth aperture, a sixth aperture and a seventh aperture;
a first valve member oriented within the first internal cavity, the first valve member defining a first valve fluid passageway;
a second valve member oriented within the second internal cavity, the second valve member defining a second valve fluid passageway;
a rotatable spindle coupled to the first valve member and the second valve member, the first valve member and the second valve member arranged to rotate with said rotatable spindle.
wherein when the rotatable spindle is in a first stop position, the first valve fluid passageway is oriented to allow fluid flow between the first aperture and the second aperture, and the second valve fluid passageway is oriented to allow fluid flow between the fourth aperture and the fifth aperture.

17. The valve assembly of claim 16, wherein when the rotatable spindle is in a second stop position, the second valve fluid passageway is oriented to allow fluid flow between the fifth aperture and the sixth aperture.

18. The valve assembly of claim 16, wherein when the rotatable spindle is in a third position, the first valve fluid passageway is oriented to allow fluid flow between the first aperture and the third aperture, and the second valve fluid passageway is oriented to allow fluid flow between the sixth aperture and the seventh aperture.

19. A method of recovering refrigerant comprising:
providing a recovery device comprising an intake port, a discharge port, a compressor, a condenser and a valve manifold assembly having a first valve portion, a second valve portion and a rotatable spindle constructed and arranged to control fluid flow through the first valve portion and the second valve portion;
connecting a fluid port of an external device having refrigerant to said intake port;
connecting said discharge port to a storage device;
placing said rotatable spindle at a first stop position and operating said recovery device to move refrigerant from said external device to said storage device;
placing said rotatable spindle at a second stop position and operating said recovery device to prepurge refrigerant from said recovery device.

20. The method of claim 19, further comprising placing said rotatable spindle at a third stop position and operating said recovery device to purge refrigerant from said recovery device.
